(19) United States
(12) Patent Application Publication
(10) Pub. No.: US 2015/0342872 A1
(43) Pub. Date: Dec. 3, 2015

(54) Use of Paclitaxel Particles

(71) Applicant: CRITITECH, INC., Lawrence, KS (US)

(72) Inventors: Stephen K. WILLIAMSON, Kansas City, MO (US); Charles J. DECEDUE, Lawrence, KS (US); Steven A. FONTANA, Wilmington, NC (US); Michael BALTEZOR, Lawrence, KS (US)

(73) Assignee: CRITITECH, INC., Lawrence, KS (US)

(21) Appl. No.: 14/546,221

(22) Filed: Nov. 18, 2014

Related U.S. Application Data
(60) Provisional application No. 62/006,215, filed on Jun. 1, 2014.

(51) Int. Cl. A61K 9/00; A61K 31/337

(52) U.S. Cl. CPC A61K 9/0019; A61K 31/337

(57) ABSTRACT

A method of administering a suspension of paclitaxel particles into the peritoneum is provided. A corresponding method of treating a disease, disorder or condition that is therapeutically responsive to paclitaxel is also provided. The methods provide improvements over comparator formulations containing an equivalent dose of paclitaxel.

The present application claims the benefit of Provisional application U.S 62/006,215 filed Jun. 1, 2014, the entire contents of which are hereby incorporated by reference.

### FIELD OF THE INVENTION

The present invention concerns methods of administering paclitaxel particles for the treatment of a disease, disorder or condition that is therapeutically responsive to paclitaxel. More particularly, the invention concerns intraperitoneal administration of a suspension containing paclitaxel particles.

### BACKGROUND OF THE INVENTION

Peritoneal carcinomatosis associated with Müllerian, GI tract cancers, mesothelioma and other malignancies cause significant morbidity and mortality. The treatment options for these diseases are limited however intraperitoneal therapy has surfaced as a treatment modality with significant potential. Clinical trials have confirmed the safety, pharmacokinetic advantage, and survival advantage for a number of agents delivered by the intraperitoneal route. Taxanes have broad antitumor activity and previous studies indicate a potential therapeutic advantage with IP administration. IP therapy is an attractive option for patients with IP carcinomatosis as many of these malignancies remain confined IP. Agents whose plasma clearance rates substantially exceed their rates of uptake from the peritoneal cavity are especially suited for IP administration. The primary toxicity associated with the IP administration of paclitaxel is transient neutropenia and abdominal pain; both appear to be dose related and it is postulated the abdominal pain is caused by the excipients CREMOPHOR® EL and ethanol that are required to overcome low drug solubility.

Paclitaxel is widely used as an anticancer drug; however, chemotherapeutic results have met with limited success in treating specific cancer types and improved methods of administration and treatment continue to be needed.

Intraperitoneal administration of paclitaxel is known; however, almost all such methods require the use of surfactants, e.g. GELUCIRE® (polyethylene glycol glycerides composed of mono-, di- and triglycerides and mono- and diesters of polyethylene glycol; Gattefosse, France) or CREMOPHOR® (polyethoxylated castor oil; BASF, Germany), or polymeric or protein excipients within which the paclitaxel is embedded or encapsulated.

According to the product literature, the commercial product ABRAXANE® (Abraxis Bioscience, L.L.C., Celgene Corporation, Summit, N.J.) contains paclitaxel protein bound particles (mean particle size about 130 nanometers) in a suspension. It is supplied in a 20 ml vial containing 100 mg of paclitaxel bound to 900 mg of human albumin (containing sodium caprylate and sodium acetyltryptophanate). The reconstituted suspension contains 5 mg of paclitaxel/mL. The pharmacokinetics of total paclitaxel following 30 and 180-minute IV (intravenous) infusions of ABRAXANE® at dose levels of 80 to 375 mg/m² were determined in clinical studies. Dose levels of mg/m² refer to mg of paclitaxel in ABRAXANE®. Following intravenous administration of ABRAXANE®, paclitaxel plasma concentrations declined in a biphasic manner, the initial rapid decline representing distribution to the peripheral compartment and the slower second phase representing drug elimination. The terminal half-life was approximately 27 hours. The drug exposure (AUCs) was dose proportional over 80 to 375 mg/m2 and the pharmacokinetics of paclitaxel were independent of the duration of ABRAXANE® administration. At the dose of 260 mg/m² for metastatic breast cancer, the mean maximum concentration of paclitaxel, which occurred at the end of the infusion, was 18,741 ng/mL. The mean total clearance was 15 L/hr/m². The mean volume of distribution was 632 L/m2 indicating extensive extravascular distribution and/or tissue binding of paclitaxel The pharmacokinetic data of 260 mg/m² ABRAXANE® administered over a 30-minute infusion was compared to the pharmacokinetics of 175 mg/m² paclitaxel injection over a 3-hour infusion. The clearance was larger (43%) and the volume of distribution was also higher (53%) for ABRAXANE® than for paclitaxel injection. Differences in the maximum concentration (Cmax) and dose-corrected Cmax reflected differences in total dose and rate of infusion. There were no differences in terminal half-lives.

According to the product literature, the commercial product TAXOL® (HG Speciality Pharma Corp.) contains paclitaxel dissolved in CREMOPHOR® and ethanol and further contains citric acid. It is supplied as a clear, viscous, non-aqueous concentrate, in 30 mg/5 mL to 30 mg/50 mL multi dose vials. Pharmacokinetic parameters of IV-administered paclitaxel following 3- and 24-hour infusions of TAXOL® at dose levels of 135 and 175 mg/m² were determined in a Phase 3 randomized study in ovarian cancer patients and are summarized in the following table:

TABLE 1
Summary of Pharmacokinetic Parameters - Mean Values

| | Infu- | | | | | |
|---|---|---|---|---|---|---|
| Dose | sion | N | Cmax | AUC (0-∞) | T- | CLT |
| (mg/ | Dura- | (pa- | (ng/ | (ng · h/ | HALF | (L/h/ |
| m2) | tion (h) | tients) | mL) | mL) | (h) | m2) |
| 135 | 24 | 2 | 195 | 6300 | 52.7 | 21.7 |
| 175 | 24 | 4 | 365 | 7993 | 15.7 | 23.8 |
| 135 | 3 | 7 | 2170 | 7952 | 13.1 | 17.7 |
| 175 | 3 | 5 | 3650 | 15007 | 20.2 | 12.2 |

Cmax = Maximum plasma concentration
AUC (0-∞) = Area under the plasma concentration-time curve from time 0 to infinity
CLT = Total body clearance

It appeared that with the 24-hour infusion of paclitaxel, a 30% increase in dose (135 mg/m2versus 175 mg/m2) increased the Cmax by 87%, whereas the AUC (0-∞) remained proportional. However, with a 3-hour infusion, for a 30% increase in dose, the Cmax and AUC (0-∞) were increased by 68% and 89%, respectively. The mean apparent volume of distribution at steady state, with the 24-hour infusion of paclitaxel, ranged from 227 to 688 L/m2, indicating extensive extravascular distribution and/or tissue binding of paclitaxel. The pharmacokinetics of paclitaxel were also evaluated in adult cancer patients who received single doses of 15-135 mg/m2 given by 1-hour infusions (n=15), 30-275 mg/m2 given by 6-hour infusions (n=36), and 200-275 mg/m2 given by 24-hour infusions (n=54) in Phase 1 & 2 studies. Values for CLT and volume of distribution were consistent with the findings in the Phase 3 study. The pharmacokinetics of paclitaxel in patients with AIDS-related Kaposi's sarcoma have not been studied. A common dosing regimen is 175 mg/m² delivered over 3 hours every 3 weeks for 4 cycles in combination with other chemotherapy.

CREMOPHOR® (Macrogolglycerol Ricinoleate Ph.Eur; Polyoxyl 35 Castor Oil USP) is a synthetic non-ionic surfactant which is a mixture (CAS number 61791-12-6): the major component is the material in which the hydroxyl groups of the castor oil triglyceride have been ethoxylated with ethylene oxide to form polyethylene glycol ethers. Minor components are the polyethyelene glycol esters of ricinoleic acide, polyethyelene glycols and polyethyelene glycol ethers of glycerol.

GELUCIRE® is a non-ionic water dispersible surfactant composed of PEG-esters, a small glyceride fraction and free PEG (lauryl macrogol glycerides (EP), lauryl polyoxyl glycerides (NF), lauroyl polyoxylglycerides USFDA IIG).

U.S. Pat. No. 8,221,779 B2 to Jonas et al. discloses an aqueous suspension containing particles of paclitaxel and an aqueous carrier. A method of treating neoplastic cell growth and proliferation in mice is disclosed; however, there is no disclosure regarding suitable dosing regimens for or particular methods of intraperitoneal administration to humans.

### SUMMARY OF THE INVENTION

The present invention seeks to overcome some or all of the disadvantages inherent in the art. The invention provides a method of administering paclitaxel in a suspension comprising paclitaxel particles in a carrier. Methods of treating a disease, disorder or condition associated with excessive cell proliferation or of treating a disease, disorder or condition that is therapeutically responsive to paclitaxel are also provided.

The method of the invention provides substantially improved pharmacokinetic parameters, improved control of paclitaxel plasma and intraperitoneal (IP) fluid concentration, improved plasma and IP fluid trough levels after 14 days, 21 days or 28 days after completion of administration of a single dose, a more level plasma concentration profile after 14 days, 21 days or 28 days after completion of administration of a dose, increased time to progression of the disease, disorder or condition, increased survival rate of subjects in a patient population, and/or improved therapeutic efficacy as compared to other dosage forms of paclitaxel administered intraperitoneally on a dose equivalent basis.

IP administration of paclitaxel particles according to the invention provides greater concentrations of paclitaxel within the peritoneal cavity compared to intravenous administration. Nanoparticulate paclitaxel will undergo prolonged dissolution resulting in greater paclitaxel concentrations at the tumor site for a longer period of time. Substantially less systemic exposure to paclitaxel will occur as compared to intravenous paclitaxel thus, reducing the risk of systemic toxicity. The methods of the invention may provide benefit to patients with (optimally) debulked disease due to greater and prolonged tumor exposure to paclitaxel. For the same reason, patients with tumors which typically do not respond well to IV paclitaxel (e.g., pancreatic, colorectal cancers) may experience a clinical benefit.

An aspect of the invention provides a method of administering paclitaxel comprising:
administering intraperitoneally to a subject in need thereof a suspension comprising paclitaxel particles and an aqueous carrier, wherein the paclitaxel is administered at a dose of at least 175 mg/m², and the maximum tolerated dose of paclitaxel is at least 275 mg/m².

Another aspect of the invention provides a method of administering paclitaxel comprising:
administering intraperitoneally to a subject in need thereof a suspension comprising paclitaxel particles and an aqueous carrier, wherein the paclitaxel is administered at a dose of at least 175 mg/m² and the resulting plasma concentration of paclitaxel is more than 2 ng/mL.

Another aspect of the invention provides a method of administering paclitaxel comprising:
administering intraperitoneally to a subject in need thereof a suspension comprising paclitaxel particles and an aqueous carrier, wherein the paclitaxel is administered at a dose ranging from 175 mg/m² to 275 mg/m², and the resulting mean or average plasma concentration of paclitaxel ranges from 2 ng/mL to 11 ng/mL during a two-week post-dosing period following completion of administration during a dosing period.

Another aspect of the invention provides a method of administering paclitaxel comprising:
administering intraperitoneally to a subject in need thereof a suspension comprising paclitaxel particles and an aqueous carrier, wherein the paclitaxel is administered in a single dose of at least 175 mg/m², and the minimum peritoneal fluid residual concentration of paclitaxel at 28 days following such administration during a dosing period is at least about 1000 ng/mL.

Another aspect of the invention provides a method of administering paclitaxel comprising:
administering intraperitoneally to a subject in need thereof a suspension comprising paclitaxel particles and an aqueous carrier, wherein the ratio of paclitaxel concentration in intraperitoneal fluid to paclitaxel concentration in plasma is greater than 1000 after completion of administration during a dosing period.

Other aspects of the invention provide methods of treating a disease, disorder or condition, having an etiology associated with excessive cell proliferation, that correspond to the methods of administering paclitaxel as described herein.

Some embodiments of the invention include those wherein: 1) the paclitaxel particles are uncoated; 2) the paclitaxel particles are not embedded, contained, enclosed or encapsulated within a solid excipient; 3) the paclitaxel particles are not microspheres or microcapsules containing paclitaxel and an excipient; 4) the paclitaxel particles exclude an added excipient; 5) the paclitaxel particles comprise at least 95% paclitaxel; 6) the paclitaxel particles consist of or consist essentially of substantially pure paclitaxel; or 7) a combination of one or more thereof.

In some embodiments, the invention requires an aqueous carrier. The carrier can comprise buffering agent, osmotic salt and/or surfactant in water. The aqueous carrier can exclude particular surfactants such as GELUCIRE® and CREMOPHOR®, as described herein. In some embodiments, the suspension excludes a polymeric excipient or a protein excipient within which paclitaxel is embedded, retained, enclosed or encapsulated.

Some embodiments of the invention include those wherein: 1) the paclitaxel particles have an average particle size in the range of 500 to 2,000 nanometers (number distribution average as measured by single particle counting); 2) the paclitaxel particles have a size distribution as follows D10≧400 nm; D90≦1,500 nm; 3) the average particle size is measured by single particle counting such as with a PSS-Nicomp AccuSizer 780; 4) the particle size distribution is measured by single particle counting such as with a PSS-Nicomp AccuSizer 780; 5) the paclitaxel particles have a number mean particle diameter about 1,000 nm or volume mean particle diameter about 4,500 nm; or 6) a combination of one or more thereof.

Some embodiments of the invention include those wherein: 1) the suspension comprises water and optionally one or more excipients selected from the group consisting of buffering agent, tonicity modifier, surfactant, demulcent, viscosity modifier, antioxidant, alkalinizing agent, acidifying agent, antifoaming agent, and osmotic agent; 2) the suspension excludes a solid excipient within which the paclitaxel is contained; 3) the suspension excludes polyethylene glycol glycerides composed of mono-, di- and triglycerides and mono- and diesters of polyethylene glycol, and polyethoxylated castor oil; 4) the suspension excludes an organic solvent; 5) the suspension comprises paclitaxel particles, water, buffer and salt; or 6) a combination of one or more thereof.

Some embodiments of the invention include those wherein: 1) a dose of the suspension is administered in one or more portions during a dosing period; 2) the dosing period ranges from 0.1 hours to 7 days, 0.1 hours to 5 days, 0.1 hours to 3 days, 0.1 hours to 2 days or 0.1 hours to 1 day; 3) the dosing period is at least 5 min; 4) the suspension is administered by perfusion or as a bolus into the peritoneal cavity; 5) the dose of suspension is diluted with a volume of 0-2000 mL, 0-1000 mL, or 0-500 mL of liquid carrier prior to administration; 6) administration of the suspension is initiated after removal of ascites fluid from the peritoneal cavity; 7) the dose is administered in a single administration during a dosing period of 24 hours or less; 8) the dose is administered as an undivided dose; 9) the entire dose is administered in a single administration either as a bolus or by infusion; or 10) a combination of one or more thereof.

Paclitaxel particles are administered during a dosing period that is followed by a post-dosing period. The suspension is not administered during the post-dosing period. A dosing cycle comprises a dosing period and post-dosing period. A treatment period comprises at least one or plural dosing cycles. Some embodiments of the invention include those wherein: 1) a treatment period comprises two or more dosing cycles; 2) a post-dosing period is at least 14 days, at least 21 days, at least 28 days, at least 35 days long; 3) a treatment period is at least 2 months, at least 3 months, at least 4 months, at least 5 months, at least 6 months, at least 9 months, at least 1 year; 4) a treatment period comprises at least 3, at least 4, at least 5, at least 6, at least 9, at least 12, at least 15, at least 18 or at least 24 dosing cycles; or 5) a combination of any of the above.

The invention provides substantial improvements over other formulations administered intraperitoneally to a subject in need thereof. Some embodiments of the invention include those wherein: 1) the invention provides increased 28-day IP fluid trough concentration of paclitaxel as compared to IP administration of an equivalent dose of a comparator formulation; 2) the invention provides increased 28-day plasma trough concentration of paclitaxel as compared to IP administration of an equivalent dose of a comparator; 3) the invention provides reduced adverse effects as compared to IP administration of an equivalent dose of a comparator suspension; 4) the invention provides increased IP fluid concentration of paclitaxel as compared to IP administration of an equivalent dose of a comparator formulation when measured at about 165-170 or about 168 hours after completion of the dosing period; 5) the invention provides increased plasma fluid concentration of paclitaxel as compared to IP administration of an equivalent dose of a comparator formulation; 6) a comparator formulation comprises a suspension comprising a liquid carrier within which are suspended particles, spheres, capsules, beads or pellets comprising paclitaxel and at least one excipient; 7) a comparator formulation comprises a solution comprising paclitaxel and a liquid carrier within which the paclitaxel is dissolved; 8) a comparator formulation further comprises GELUCIRE® or CREMOPHOR®; 9) a comparator formulation comprises paclitaxel coated with or containing a polymer; or 10) a combination of any of the above.

The invention includes all combinations of the aspects, embodiments and sub-embodiments disclosed herein.

### BRIEF DESCRIPTION OF THE FIGURES

The following figures form part of the present description and describe exemplary embodiments of the claimed invention. The skilled artisan will be able to practice the invention without undue experimentation in light of these figures and the description herein.

FIG. 1 depicts charts of the plasma and peritoneal pharmacokinetics of paclitaxel following intraperitoneal administration of an aqueous suspension containing paclitaxel particles at a dose of 175 mg/m² in an individual patient during two dosing cycles for the study described herein.

FIG. 2 depicts a chart of the time dependent change of peritoneal fluid paclitaxel concentrations for each dose level for the study described herein.

FIG. 3 depicts a chart of the time dependent change of plasma paclitaxel concentrations for each dose level for the study described herein.

### DETAILED DESCRIPTION OF THE INVENTION

Aspects and embodiments of the invention include a method of administering paclitaxel intraperitoneally, a method of treating a disease, disorder or condition that is therapeutically responsive to paclitaxel, and a method of treating a disease, disorder or condition having an etiology associated with excessive cell proliferation.

As used herein and unless otherwise specified, the term “paclitaxel particles” refers to particles of paclitaxel that do not include an added excipient. Paclitaxel particles are different than “particles containing paclitaxel”, which are particles that contain paclitaxel and at least one added excipient. Paclitaxel particles exclude a polymeric, wax or protein excipient and are not embedded, contained, enclosed or encapsulated within a solid excipient. Paclitaxel particles may, however, contain impurities and byproducts typically found during preparation of paclitaxel. Even so, paclitaxel particles comprise at least 95%, at least 97%, at least 99% or 100% paclitaxel, meaning the paclitaxel particles consist of or consist essentially of substantially pure paclitaxel.

The paclitaxel particles will typically possess an average or mean particle size in the range of 0.2-5, 0.4-3 or 0.5-1.4 microns. The particles will exhibit particle size distributions characterized as follows:

| Form # | D10 (microns) | D50 (microns) | D90 (microns) |
|---|---|---|---|
| 1 | ≦0.1 | 0.4 ≧ and ≦4 | ≧7 |
| 2 | 0.2-0.5 | 0.4-2 | 2.5-5 |
| 3 | 0.4-0.5 | 0.5-1.4 | 0.8-2.4 |

Paclitaxel particles can be prepared by any suitable method. An exemplary method is crystallization or precipitation of paclitaxel from a supercritical fluid, e.g. supercritical carbon dioxide, or from a cryogenic fluid or a multiphase fluid. Spray-drying is also suitable. Suitable methods for preparation of paclitaxel particles are disclosed in U.S. Pat. No. 8,221,779, U.S. Pat. No. 6,113,795, U.S. Pat. No. 5,833,891 (without the coating material), U.S. Pat. No. 5,874,029 (without the coating material), U.S. Pat. No. 7,744,923, PCT Application No. PCT/US2014/028507, filed Mar. 14, 2014, and copending U.S. application Ser. No. 13/911,700, filed Jun. 6, 2013, the entire disclosures of which are hereby incorporated by reference.

A subject (patient) having a disease, disorder or condition that is therapeutically responsive to paclitaxel will exhibit a reduced number of symptoms, reduced severity of symptoms, improved overall health, lack of progression of the disease, disorder or condition, or remission or regression of the disease, disorder or condition. Exemplary diseases, disorder or conditions include those having an etiology associated with excessive cell proliferation. Specific examples include cancer, carcinoma or tumor, in particular breast, ovarian, lung, bladder, prostate, melanoma, esophageal, metastatic breast cancer, non-small cell lung cancer, adenocarcinoma of the pancreas, as well as other types of solid tumor cancers. It can also been used in treating Kaposi's sarcoma.

The suspension of the invention comprises paclitaxel particles and a liquid carrier. The liquid carrier can be aqueous. The suspension excludes a solid excipient within which the paclitaxel is contained and excludes GELUCIRE® (polyethylene glycol glycerides composed of mono-, di- and triglycerides and mono- and diesters of polyethylene glycol), and CREMOPHOR® (polyethoxylated castor oil), and an organic solvent.

Even though the paclitaxel particles do not include an added excipient, the liquid carrier of the suspension can comprise water and optionally one or more excipients selected from the group consisting of buffer, tonicity adjusting agent, preservative, demulcent, viscosity modifier, osmotic agent, surfactant, antioxidant, alkalinizing agent, acidifying agent, antifoaming agent, and colorant. For example, the suspension can comprise paclitaxel particles, water, buffer and salt. It optionally further comprises a surfactant. In some embodiments, the suspension consists essentially of or consists of water, paclitaxel particles suspended in the water and buffer. The suspension can further contain an osmotic salt.

The suspension can comprise one or more surfactants. Suitable surfactants include by way of example and without limitation polysorbates, lauryl sulfates, acetylated monoglycerides, diacetylated monoglycerides, and poloxamers.

The suspension can comprise one or more tonicity adjusting agents. Suitable tonicity adjusting agents include by way of example and without limitation, one or more inorganic salts, electrolytes, sodium chloride, potassium chloride, sodium phosphate, potassium phosphate, sodium, potassium sulfates, sodium and potassium bicarbonates and alkaline earth metal salts, such as alkaline earth metal inorganic salts, e.g., calcium salts, and magnesium salts, mannitol, dextrose, glycerin, propylene glycol, and mixtures thereof.

The suspension may be formulated to be hyperosmolar (hypertonic), hyposmolar (hypotonic) or isosmolar (isotonic) with respect to the fluid(s) of the IP cavity. In some embodiments, the suspension will be isotonic with respect to fluid in the IP cavity. The osmolality of the suspension can range from about 200 to about 380, about 240 to about 340, about 280 to about 300 or about 290 mOsm/kg.

The suspension can comprise one or more buffering agents. Suitable buffering agents include by way of example and without limitation, dibasic sodium phosphate, monobasic sodium phosphate, citric acid, sodium citrate hydrochloric acid, sodium hydroxide, tris(hydroxymethyl)aminomethane, bis(2-hydroxyethyl)iminotris-(hydroxymethyl)methane, and sodium hydrogen carbonate and others known to those of ordinary skill in the art. Buffers are commonly used to adjust the pH to a desirable range for intraperitoneal use. Usually a pH of around 5 to 9, 5 to 8, 6 to 7.4, 6.5 to 7.5, or 6.9 to 7.4 is desired.

The suspension can comprise one or more demulcents. A demulcent is an agent that forms a soothing film over a mucous membrane, such as the membranes lining the peritoneum and organs therein. A demulcent may relieve minor pain and inflammation and is sometimes referred to as a mucoprotective agent. Suitable demulcents include cellulose derivatives ranging from about 0.2 to about 2.5% such as carboxymethylcellulose sodium, hydroxyethyl cellulose, hydroxypropyl methylcellulose, and methylcellulose; gelatin at about 0.01%; polyols in about 0.05 to about 1%, also including about 0.2 to about 1%, such as glycerin, polyethylene glycol 300, polyethylene glycol 400, polysorbate 80, and propylene glycol; polyvinyl alcohol from about 0.1 to about 4%; povidone from about 0.1 to about 2%; and dextran 70 from about 0.1% when used with another polymeric demulcent described herein.

The suspension can comprise one or more alkalinizing agents to adjust the pH. As used herein, the term “alkalizing agent” is intended to mean a compound used to provide an alkaline medium. Such compounds include, by way of example and without limitation, ammonia solution, ammonium carbonate, potassium hydroxide, sodium borate, sodium carbonate, sodium bicarbonate, and sodium hydroxide and others known to those of ordinary skill in the art

The suspension can comprise one or more acidifying agents to adjust the pH. As used herein, the term “acidifying agent” is intended to mean a compound used to provide an acidic medium. Such compounds include, by way of example and without limitation, acetic acid, amino acid, citric acid, nitric acid, fumaric acid and other alpha hydroxy acids, hydrochloric acid, ascorbic acid, and nitric acid and others known to those of ordinary skill in the art.

The suspension can comprise one or more antifoaming agents. As used herein, the term “antifoaming agent” is intended to mean a compound or compounds that prevents or reduces the amount of foaming that forms on the surface of the fill composition. Suitable antifoaming agents include by way of example and without limitation, dimethicone, SIMETHICONE, octoxynol and others known to those of ordinary skill in the art.

The suspension can comprise one or more viscosity modifiers that increase or decrease the viscosity of the suspension. Suitable viscosity modifiers include methylcellulose, hydroxypropyl methycellulose, mannitol and polyvinylpyrrolidone.

The suspension can comprise one or more osmotic agents such as those used for peritoneal dialysis. Suitable osmotic agents include icodextrin (a glucose polymer), sodium chloride, potassium chloride, and salts that are also used as buffering agents.

As used herein, “time to progression” is the period, length or duration of time after a disease is diagnosed (or treated) until the disease begins to worsen (such as until a tumor begins or continues to grow). It is the period of time during which the level of a disease is maintained without further progression of the disease, and the period of time ends when the disease begins to progress again. Progression of a disease is determined by “staging” a subject suffering from a cell proliferative disease prior to or at initiation of therapy. For example, the size, location and number of tumors a subject has is determined prior to or at initiation of therapy. The subject is then treated with the suspension of the invention, and the size and number of tumors are monitored periodically. At some later point in time, the size and/or number of tumors may increase, thus marking progression of the disease and the end of the “time to progression”. The period of time during which the disease did not progress, or during which biological markers for the disease did not increase, or during which the level or severity of the disease did not worsen is the “time to progression”.

It should be noted that a subject's therapeutic response can be a full or partial response at therapeutically relevant doses administered to the subject. In other words, the level of therapeutic response is determined at a dose that would not be fatal to a subject to which the suspension is administered. A therapeutically relevant dose, therefore, is a therapeutic dose at which a therapeutic response of the disease or disorder to treatment with the suspension is observed and at which a subject can be administered the suspension without an excessive amount of unwanted or deleterious side effects. A therapeutically relevant dose is non-lethal to a subject, even though it may cause some side effects in the patient. It is a dose at which the level of clinical benefit to a subject being administered the paclitaxel exceeds the level of deleterious side effects experienced by the subject due to administration of the suspension. A therapeutically relevant dose will vary from subject to subject according to a variety of established pharmacologic, pharmacodynamic and pharmacokinetic principles.

Paclitaxel particles can be present as a dry powder in a container. The container comprises one or more doses of paclitaxel particles. In some embodiments, a container comprises a single dose of paclitaxel particles. The suspension can be prepared by adding a liquid carrier to a container comprising paclitaxel particles. One or more excipients can be included in the container and/or in the liquid carrier added to the container. The suspension can be prepared by mixing paclitaxel particles ready for constitution with an appropriate liquid carrier, and optionally one or more excipients, prior to IP administration. In some embodiments, the process for preparing the suspension comprises adding a liquid carrier to a container comprising paclitaxel particles, wherein one or more excipients are optionally included in the container and/or in the liquid carrier added to the container.

The suspension can be formulated to contain 0.5-10, 1-8, 3-6 or 4-5 mg of paclitaxel particles per ml of suspension prior to administration. The suspension can be administered as is or can be diluted with a diluent, e.g. with saline water for injection optionally including a buffering agent and one or more other excipients, prior to administration. For example, the volume ratio of suspension to diluent might be in the range of 1:1-1:100 v/v or other suitable ratio. Alternatively, the suspension may also be added to a freely flowing instillation line delivering diluent (e.g. plasma or buffered saline water). For example, 50 mL of suspension might be added at rate of 1 mL/min for 50 min. into a total volume of saline water of 1 L instilled into a subject.

A concentrated suspension intended to be diluted prior to administration can contain 1-500, 1-250 or 1-100 mg of paclitaxel particles per mL of suspension. For example, a concentrated suspension containing 100 mg of paclitaxel particles per mL can be diluted with a sufficient volume of liquid carrier to form a diluted suspension containing 1-10 mg of paclitaxel particles per mL. Alternatively, a concentrated suspension can be administered to the IP cavity and then diluted by instillation of additional liquid carrier into the IP cavity.

A dose can be administered according to any dosing regimen typically used in the treatment of diseases or disorders having an etiology associated with excessive cell proliferation. The dose of paclitaxel particles administered will vary according to common principles of pharmacology and chemotherapy. Suitable doses include ≧25 mg/m², ≧50 mg/m², ≧75 mg/m², ≧82.5 mg/m², ≧100 mg/m², ≧125 mg/m², ≧150 mg/m², ≧175 mg/m², ≧200 mg/m², ≧225 mg/m², ≧250 mg/m², ≧275 mg/m² or higher.

The maximum tolerated dose (MTD) of paclitaxel particles can be determined by dose escalation or de-escalation of an initial dose according to basic pharmaceutical principles and a subject's clinical response to paclitaxel. The MTD is equal to or greater than 275 mg/m² and can be 500 mg/m² or higher.

A dosing period is that period of time during which a dose of paclitaxel particles in the suspension is administered. The dosing period can be a single period of time during which the entire dose is administered, or it can be divided into two or more periods of time during each of which a portion of the dose is administered. For example, a first portion of the dose can be administered within the first hour(s) of the dosing period, and a second portion of the dose can be administered at a later time period. In such a case, the dosing period is defined as that period of time including initial administration of the first portion through to final administration of the second portion. Accordingly, a dosing period can last from 5 min to 72 hrs, 5 min to 48 hrs or 5 min to 24 hrs. In some embodiments, the dosing period is 48 hours or less, 24 hours or less, 18 hours or less, 12 hours or less, 6 hours or less, 3 hours or less or 1 hour or less. The dosing period can be kept constant for a subject or two or more different dosing periods can be used for a subject. In some embodiments, the undivided dose is administered in a single period of time. In some embodiments, the dose is administered in a single administration.

A post-dosing period is that period of time beginning after completion of a prior dosing period and ending after initiating a subsequent dosing period. The duration of the post-dosing period may vary according to a subject's clinical response to the paclitaxel. The suspension is not administered during the post-dosing period. A post-dosing period can last at least 7 days, at least 14 days, at least 21 days, at least 28 days, at least 35 days, at least 60 days or at least 90 days or longer. Due to the high residence time of paclitaxel particles in the peritoneum following instillation of the suspension, the post-dosing period can be longer than that provided by another dosage form containing paclitaxel. The post-dosing period can be kept constant for a subject or two or more different post-dosing periods can be used for a subject.

A dosing cycle includes a dosing period and a post-dosing period. Accordingly, the duration of a dosing cycle will be the sum of the dosing period and the post-dosing period. The dosing cycle can be kept constant for a subject or two or more different dosing cycles can be used for a subject.

A treatment period comprises at least one dosing cycle and typically comprises two or more dosing cycles. For example, a treatment period can last at least 1 month, at least 2 months, at least 3 months, at least 4 months, at least 5 months, at least 6 months, at least 9 months, at least 1 year or longer, or a treatment period comprises at least 2, at least 3, at least 4, at least 5, at least 6, at least 9, at least 12, at least 15, at least 18, at least 24 or more dosing cycles.

Pre-clinical studies of a novel formulation of nanoparticulate paclitaxel in animal tumor models demonstrated superior activity and substantially reduced systemic toxicity. This allowed for significant IP doses and concentrations, yet yielded very low systemic concentrations of paclitaxel. We report here the results of a Phase 1 trial of IP administered NTX. Methods: Patients (ECOG≦2) had relapsed solid IP tumors and adequate organ function. NTX was administered IP as a bolus injection after 500 ml saline water followed by IP administration of up to 2 L of saline water. We utilized an accelerated dose escalation scheme until one DLT occurred during cycle 1, followed by a standard dose escalation (3+3 design) based on CTCAE V3 toxicities. The pharmacokinetics of IP administered NTX were characterized in plasma and ascites fluid. Secondary objectives were to define the recommended phase 2 dose of NTX, and to characterize preliminary activity and toxicity. Patients (n=21) were treated at dose levels from 50-275 mg/m² q 28 days. Primary malignancy was ovarian cancer (74%). Treatment was well tolerated at all dose levels. Common toxicities potentially related to NTX were: gastrointestinal (68%), constitutional (42%), and pain (42%). Average number of cycles received was 2 (range 1 to 6). Best response was stable disease (4 patients, 21%). Median length of disease stability was 99 days (range 85 to 151 days); the median time for study patients with stable disease was 313 days (range 142 to 740 days). All Cmax in plasma were less than 35 ng/mL, with ascites (IP) fluid Cmax generally greater than 1000 ng/mL.

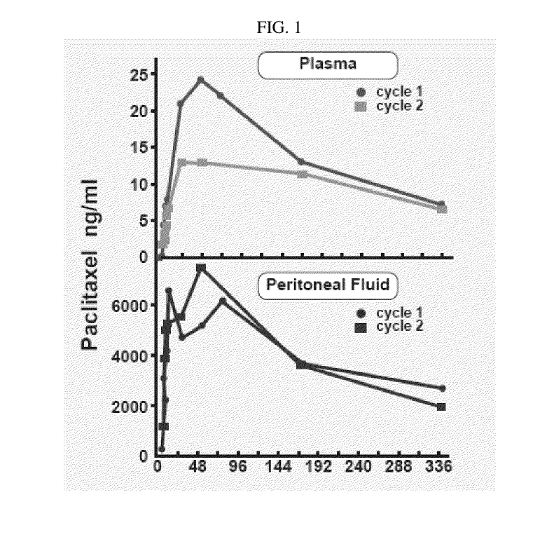

FIG. 1 depicts charts of the plasma and peritoneal pharmacokinetics of paclitaxel following intraperitoneal administration of NTX (175 mg/m²) in an individual patient during two dosing cycles. Systemic exposure to paclitaxel is minimal following intraperitoneal administration of NTX. In contrast peritoneal concentration of paclitaxel exceeds the typical therapeutic levels achieved systemically after IV paclitaxel administration. In addition, clearance of paclitaxel from the peritoneal fluid is slow.

The study was continued to include additional dosing levels and extend the treatment period and the following observations were made. Peritoneal fluid paclitaxel concentrations increased rapidly after the first treatment cycle of IP paclitaxel particles (for all dose levels combined, mean 2 hour post administration concentration was 5711 ng/mL). The individual mean maximum peak (Cmax) peritoneal fluid concentrations by dose level were 3624 ng/mL, 4588 ng/mL, 3988 ng/mL, 11247 ng/mL, 8004 ng/mL, and 23613 ng/mL, for the 50 mg/m2, 82.5 mg/m², 125 mg/m², 175 mg/m², 225 mg/m², and 275 mg/m² IP doses, respectively. The mean time to reach (Tmax) the maximum peritoneal fluid concentrations (Cmax) by dose level was 168 h, 26 h, 8 h, 29 h, 56 h, 38 h, for the 50 mg/m², 82.5 mg/m², 125 mg/m², 175 mg/m², 225 mg/m², and 275 mg/m² IP doses, respectively.

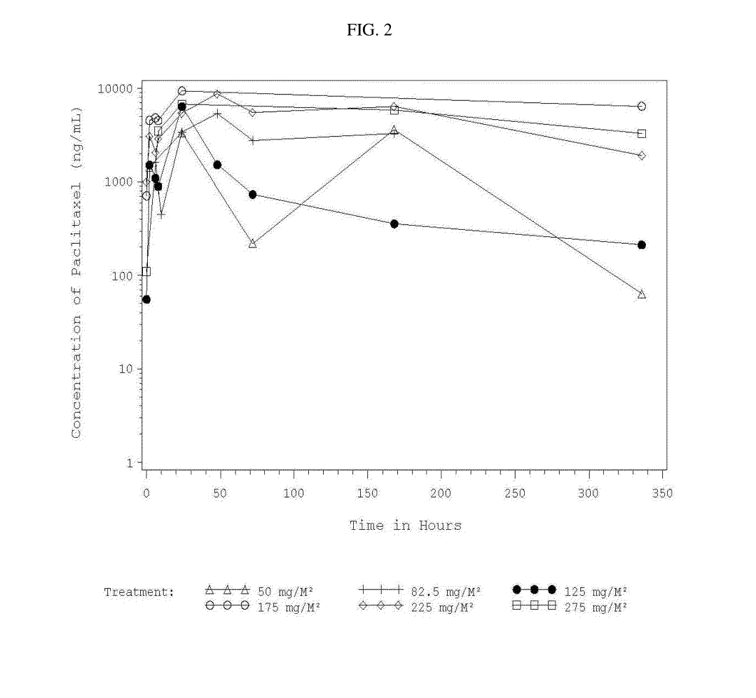

The total peritoneal cavity exposure to paclitaxel particles was not calculated. However, peritoneal fluid paclitaxel concentrations for each dose level over the sampling measurement period (approximately 336 h) remained significantly elevated over the sampling period (FIG. 2). The half-life (t1/2) and its associated elimination rate constant were not calculated for peritoneal fluid paclitaxel, but clearance occurred slowly from the peritoneal compartment. Peritoneal fluid concentrations of paclitaxel obtained prior to the IP dose of the second cycle revealed that 10 of the 12 subjects had detectable paclitaxel levels (mean 1087 ng/mL±1373.9 (SD)). After approximately 4 weeks (672 hours) peritoneal paclitaxel concentration are evident from the first IP paclitaxel particle dose. A peritoneal paclitaxel concentration was measured in all doses ≧125 mg/m²; mean paclitaxel concentrations of >1000 ng/mL were observed in the IP fluid of the peritoneal cavity for the 175 mg/m² and 225 mg/m² dose levels.

Mean trough peritoneal fluid concentrations of paclitaxel, i.e., values just prior to the second IP dose, exceed 1000 ng/mL for the 175 mg/m² and 225 mg/m² dose levels, whereas the corresponding mean plasma concentrations are less than 5 ng/mL. Trough peritoneal concentrations remained sustained, and the corresponding plasma concentrations remained low across the dose levels. After approximately 4 weeks, peritoneal paclitaxel concentration were still sustained and when combined with the 2 week peritoneal concentration-time profile indicated that paclitaxel levels remain extremely elevated and sustained over the entire dosing interval of 28 days (a dosing cycle). This elevated IP fluid trough concentration is substantially higher than can be achieved by known suspension formulations comprising particles containing paclitaxel and at least one other excipient or formulations comprising paclitaxel dissolved in solution, e.g. TAXOL®.

A mean plasma paclitaxel concentration of >4.5 ng/mL was observed 2 hours after the first treatment cycle of IP paclitaxel particles (for all dose levels combined). The individual mean maximum peak (Cₘₐₓ) plasma concentration by dose level was 6.2 ng/mL, 10.9 ng/mL, 10.8 ng/mL, 14.8 ng/mL, 13.2 ng/mL, and 12.3 ng/mL, for the 50 mg/m², 82.5 mg/m², 125 mg/m², 175 mg/m², 225 mg/m², and 275 mg/m² IP doses, respectively.

Plasma concentrations of paclitaxel obtained prior to the dose of the second cycle, i.e. prior to the second dosing period, revealed that 12 of the 14 subjects (who had pre-dose second cycle levels reported) had detectable paclitaxel levels (mean 2.6 ng/mL). Plasma paclitaxel concentrations were measured in dose levels 82.5 mg/m² to 225 mg/m²; with all paclitaxel concentrations <5 ng/mL.

The method of the invention provides a mean or average plasma concentration of paclitaxel ranging from 2 ng/mL to 11 ng/mL during a two-week post-dosing period following completion of a dosing period when paclitaxel is administered at a dose ranging from 175 mg/m² to 275 mg/m². Similarly, the method of the invention provides a mean or average plasma concentration of paclitaxel of at least 2 ng/mL when paclitaxel is administered at a dose of at least 175 mg/m². This elevated plasma concentration is substantially higher than can be achieved by known suspension formulations comprising particles containing paclitaxel and at least one other excipient or formulations comprising paclitaxel dissolved in solution, e.g. TAXOL®.

The mean time (Tₘₐₓ) to reach the maximum plasma concentrations (Cₘₐₓ) by dose level was 48 h, 46 h, 36 h, 73 h, 148 h, 53 h, for the 50 mg/m², 82.5 mg/m², 125 mg/m², 175 mg/m², 225 mg/m², and 275 mg/m² IP doses, respectively.

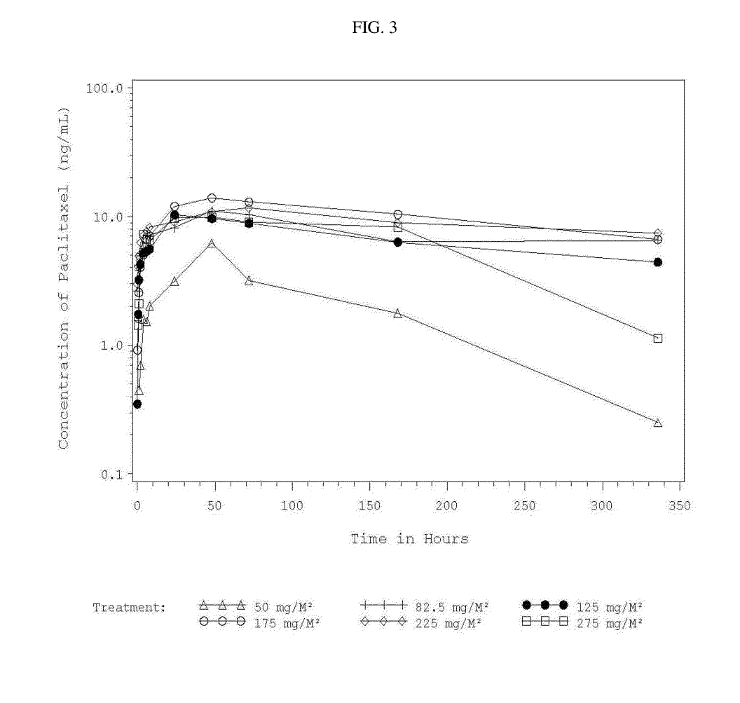

Total systemic (plasma) exposure to paclitaxel particles over the first 2 weeks of treatment (336 h) by dose level was 684 ng*h/mL, 2209 ng*h/mL, 1856 ng*h/mL, 3305 ng*h/mL, 3213 ng*h/mL, and 1735 ng*h/mL, for the 50 mg/m², 82.5 mg/m², 125 mg/m², 175 mg/m², 225 mg/m², and 275 mg/m² IP doses, respectively. The plasma paclitaxel concentrations for each dose level over the sampling period (approximately 336 h) remained low but plateaued with a slow apparent terminal clearance from the systemic compartment (FIG. 3).

Peritoneal peak exposures of paclitaxel are approximately 375 to 2000 times greater than paclitaxel peak systemic exposures. The plasma concentration-time profile resembles and parallels (at substantially lower concentrations) the rise, plateau and elimination slope observed with the peritoneal concentration-time profile. The apparent plasma elimination rate is very slow with mean t1/2 values that range from 78 h to 477 h across the different dose levels. Peritoneal paclitaxel PK characteristics indicate nanoparticle paclitaxel has a relatively small initial peritoneal distribution volume with an extremely low clearance from the peritoneal cavity. Accordingly, the method of the invention provides a ratio of paclitaxel concentration in intraperitoneal fluid to paclitaxel concentration in plasma of greater than or equal to 1000 ng/mL after completion of administration during a dosing period. This high ratio permits dosing of high amounts of paclitaxel IP while minimizing systemic exposure, thus providing reduced adverse effects caused by paclitaxel and increased direct exposure of cancers or tumors in the peritoneal cavity to paclitaxel as compared to intravenous administration of paclitaxel, e.g. as compared to intravenous administration of TAXOL®.

Paclitaxel half-lives could be determined (for a few assessments) for each dose level and were 78 h, 477 h, 180 h, 185 h, 178 h, 106 h, for the 50 mg/m², 82.5 mg/m², 125 mg/m², 175 mg/m², 225 mg/m², and 275 mg/m² IP doses, respectively.

The data indicate that IP NTX is well tolerated. A MTD (maximum tolerated dose) of at least 275 mg/m2 was observed, but the highest MTD was not reached. Pharmacokinetic data demonstrate significant, persistent IP exposure to paclitaxel with minimal systemic exposure.

Tumor assessments and progression data by dose level, progression and survival data, time to progression and time to death summary, and death report summary by subject was determined. Sixteen subjects had CA125 assessments; with 8 subjects having an increase in their levels, 3 subjects having a decrease in their levels, 1 subject with no change in their levels and 4 subjects having no assessments. 4 subjects with a reduction or no change in CA125 levels was observed in the 175 mg/m² (3 subjects) and 225 mg/m² (1 subject) dose levels. Five subjects had localized tumor progression, 7 subjects with regional or nodal progression, and 6 subjects with distant progression. Seventeen deaths were recorded over the course of the study due to the subjects' primary cancer. Time to tumor or clinical disease progression was longest in the 175 mg/m² dose level (median 81 days). Time to death was longest in the 82.5 mg/m² dose level (median 332 days). These data are consistent with the refractory nature and clinical status of these subjects at study enrollment.

Further, this trial suggests NTX when administered by the IP route will provide paclitaxel peritoneal cavity concentrations several magnitudes greater than the minimal concentration (>0.1 micromol/L) required to induce microtubule bundling and cytotoxic effects, especially for patients with ovarian cancer. The extremely low clearance from the peritoneal cavity provides sustained paclitaxel concentrations over a long duration which may be optimal for treating slow growing neoplasms with this cell-cycle specific agent.

In order to establish the differences in performance between the instant invention and other paclitaxel-containing formulations, intravenous administration of TAXOL® was evaluated alongside intraperitoneal administration of the suspension of the invention. It was determined that TAXOL® exhibits extremely high initial plasma concentrations of paclitaxel followed by a rapid clearance within about 4-5 days, whereby there is no detectable level of paclitaxel in the plasma following 1 week of administration. One the other hand, IP administration of the suspension according to the invention provided an initial 25-100-fold reduction in the initial plasma concentration but also provided a sustained and quantifiable trough level of paclitaxel even up to 28 days after administration.

The clinical data described herein indicates the suspension can provide a mean or average plasma concentration of paclitaxel ranging from 2 ng/mL to 11 ng/mL during a two-week post-dosing period following completion of administration during a dosing period when the paclitaxel is administered at a dose ranging from 175 mg/m² to 275 mg/m². This elevated plasma concentration is substantially higher than can be achieved by known suspension formulations comprising particles containing paclitaxel and at least one other excipient.

The methods of the invention can further comprise administering one or more other pharmaceutically active ingredients. These other active ingredients can be included in the suspension or can be administered to a subject in another dosage form. In some embodiments, the other active ingredient(s) is included in the suspension. In other embodiments, the other active ingredient is included in another dosage form that is administered to the subject. In some embodiments, the other active ingredient is also present as particles that exclude an added excipient, e.g. a substantially pure form of the active ingredient.

Preferred other active ingredients will cooperate with paclitaxel to ameliorate the disease, disorder or condition being treated, reduce the number or intensity of adverse effects caused by paclitaxel, enhance the therapeutic benefit of paclitaxel, and/or otherwise enhance the clinical benefit provided by paclitaxel. In some embodiments, the drugs will cooperate additively or synergistically. Suitable other active ingredients include platins (platinum-based drugs), quinoline alkaloids, epithilones, Vinca alkaloids, camptothecin analogs, nucleoside analogs, 3-amino-5-hydroxybenzoic acid analogs, epipodophyllotoxins and others known in the art for the treatment of a disease, disorder or condition having an etiology associated with excessive cell proliferation. Specific other active ingredients include carboplatin, cisplatin, oxaliplatin, camptothecin, topotecan (TPT), irinotecan (CPT-11), gemcitabine, vinblastine, vincristine, vindesine, vinorelbine, etoposide, teniposide, mytomycin C, rifamycin, and ansamycin.

The amount of other active ingredient included in the suspension will be whatever amount is therapeutically effective and will depend upon a number of factors, including the identity and potency of the chosen active ingredient, the disorder being treated, the health of the subject being treated and other such factors common to the pharmaceutical industry for prescription of drugs to a subject. The active ingredients will generally be administered according to their known dosing regimens such as those disclosed in the Pharmaceutical Desk Reference or those recognized as suitable by the Food and Drug Administration (USA), European Medicines Agency (Europe), National Institute of Health Sciences (Japan), and National Administration of Drugs, Food, and Medical Technology (Administración Nacional de Medicamentos, Alimentos y Tecnología Médica, Argentina).

In view of the above description and the examples below, one of ordinary skill in the art will be able to practice the invention as claimed without undue experimentation. The foregoing will be better understood with reference to the following examples that detail certain assemblies and methods according to the present invention. All references made to these examples are for the purposes of illustration. The following examples should not be considered exhaustive, but merely illustrative of only a few of the many embodiments contemplated by the present invention.

### Example 1

A suspension containing paclitaxel particles can be prepared with the following ingredients in the amounts indicated.

| INGREDIENT | AMOUNT | |
|---|---|---|
| paclitaxel particles | 0.1 to 1% | wt. |
| Buffer, to pH 6-7.8 | 0.01-0.5M | |
| Water | Q.S. to 100% | wt. |
| Tonicity adjusting agent | 0.1 to 1.2% | wt. |
| Demulcent | 0-5% | wt. |
| Viscosity modifier | 0.-5% | wt. |
| Surfactant | 0-1% | wt. |

Paclitaxel particles can be prepared according to the process described in U.S. Pat. Nos. 5,833,891 and 6,113,795. Paclitaxel can be recrystallized to an average particle size and a particle size distribution as described herein such that 95% wt of the particles were below 1.7 micron in size as determined by single particle light obscuration counting.

The ingredients are placed in water and mixed to form a suspension. The suspension can be administered as is or can be diluted prior to administration.

### Example 2

A suspension containing paclitaxel particles can be prepared with the following ingredients in the amounts indicated.

| INGREDIENT | AMOUNT | |
|---|---|---|
| paclitaxel particles | 0.1-1% | wt. |
| Buffer, to pH 6-7.5 | 0.1M | |
| Tonicity modifier | 0.5-1% | wt. |
| Water | Q.S. to 100% | wt. |
| Surfactant | 0-1% | wt. |
| Demulcent | 0-5% | wt. |
| Viscosity modifier | 0-5% | wt. |

The ingredients are placed in water and mixed to form a suspension. The suspension can be administered as is or can be diluted prior to administration.

### Example 3

A suspension containing paclitaxel particles can be prepared with the following ingredients in the amounts indicated.

| INGREDIENT | AMOUNT |
|---|---|
| paclitaxel particles | (5-10 mg/mL) 0.5-1% wt |
| Phosphate buffer, to pH 6.5-7.4 | 0.05-0.2M |
| NaCl | 0.75-1% wt. |
| Polysorbate 80 | 0-0.1% wt. |
| Water | Q.S. to 100% |
| Demulcent | 0-5% wt. |

The ingredients are placed in water and mixed to form a suspension. The suspension can be administered as is or can be diluted prior to administration.

### Example 4

A suspension containing paclitaxel particles can be prepared with the following ingredients in the amounts indicated.

| INGREDIENT | AMOUNT (% WT.) |
|---|---|
| Paclitaxel particles | (7.5-10 mg/mL) 0.75-1% wt |
| Sodium phosphate buffer (pH 6.8-7.4) | 0.05-0.15M |
| Water | Q.S. to 100% |
| NaCl | 0.75-0.9% wt. |
| Mannitol | 0-5% wt. |
| Methylcellulose | 0-5% wt. |
| Surfactant | 0-1% wt. |

The solid ingredients are mixed and then placed in water to form a suspension. Other non-solid ingredients are added to the suspension as needed. The suspension can be administered as is or can be diluted prior to administration.

### Example 5

A concentrated suspension containing paclitaxel particles can be prepared with the following ingredients in the amounts indicated.

| INGREDIENT | AMOUNT |
|---|---|
| paclitaxel particles | >1% up to 10% wt. |
| Buffer, to pH 6-7.5 | 0.1-5M |
| Water | Q.S. to 100% wt. |
| Tonicity adjusting agent | 1 to 10% wt. |

The solid ingredients are mixed and then placed in water to form a suspension. Other non-solid ingredients are added to the suspension as needed. One or more excipients not listed above can be added to the powder of paclitaxel particles, to the liquid carrier or to the suspension. The suspension is diluted as needed prior to administration to achieve a concentration of paclitaxel particles suitable for IP administration.

### Example 6

A suspension containing paclitaxel particles can be prepared with the following ingredients in the amounts indicated.

| INGREDIENT | AMOUNT (% WT.) | |
|---|---|---|
| Paclitaxel particles (average particle size in | 0.5-0.75% | wt |
| the range of 0.4-1.4 microns) | | |
| Sodium Phosphate (pH 6.8-7.4) | 0.01M | |
| NaCl | 0.75-0.9% | wt. |
| Polysorbate 80 | 0.1% | wt. |
| Water | Q.S. to 100% | wt. |
| Demulcent | 0-5% | wt. |
| Viscosity modifier | 0-5% | wt. |

The solid ingredients are mixed and then placed in water to form a suspension. Other non-solid ingredients are added to the suspension as needed. The suspension can be administered as is or can be diluted prior to administration.

### Example 7

A suspension containing paclitaxel particles (NTX®) was evaluated in humans according to Phase I study Protocol No. HSC#11140 entitled “Intraperitoneal Nanoparticle paclitaxel in Patients with Peritoneal Malignancies”. Solid tumors, ovarian cancer and other tumor types principally localized to the peritoneal cavity were treated. The study was conducted according to DHSS and USFDA regulations and guidelines.

The objectives of the study were to: a) evaluate the pharmacokinetics (PK) of intraperitoneally administered nanoparticle paclitaxel (NTX) in patients with refractory malignancies principally confined to the peritoneal cavity; b) compare the paclitaxel PK following intaperitoneal administration of NTX to literature data obtained following intravenous and intraperitoneal paclitaxel (TAXOL); c) determine the maximally tolerated dose (MTD) and to assess the qualitative and quantitative toxicities of NTX; and d) evaluate in a preliminary manner for any anti-tumor activity using RECIST criteria.

Bulk paclitaxel was provided by Natural Pharmaceuticals, Inc. (Beverly, Mass.). Bulk paclitaxel is converted to nanoparticles using a PCA technique, which employs super-critical carbon dioxide and acetone to generate paclitaxel nanoparticles within a well-characterized particle-size distribution: median particle size of 0.75 microns, D10 of 0.4 microns, D90 of 1.5 microns.

The suspension was prepared from paclitaxel nanoparticles by reconstitution with water for injection containing 0.9% NaCl to provide a concentration of 5 mg paclitaxel/mL of suspension. The suspension was administered IP as a bolus injection after 500 ml saline followed by IP administration of up to 2 L of saline as tolerated. Fluid was not removed from the peritoneum except for pharmacokinetic sampling.

### Administration:

Any appreciable ascites was drained from the peritoneal cavity prior to dialysate administration. The peritoneal catheter and study medication administration tubing were clamped off to prevent air from entering the peritoneal catheter when connecting or disconnecting the administration tubing. The dialysate container was attached to the administration tubing, suspend above the patient, and the clamp released to allow the solution to run through the tubing to expel air to reduce the amount of air entering the peritoneal cavity.

The access point over the port could be infiltrated with a local or topical anesthetic prior to access. The skin over the port site was thoroughly cleansed with Betadine, and the fluid tubing attached to the peritoneal catheter access port with a 19 gauge Huber style needle using sterile technique. The peritoneal catheter connector tubing/catheter cap were thoroughly cleansed with Betadine and connected to the dialysate tubing. The dialysate was allowed to flow into the peritoneal cavity by gravity drainage. After administration of 0.5 L of 0.9% Sodium Chloride for Injection, USP, the study medication (chemotherapy solution) was administered be a bolus injection into the tubing.

Paclitaxel tubing was used with the filter removed to prevent trapping of study medication particles during IP injection. The appropriate dose of study medication was drawn into 60 mL syringe(s). Following the injection additional saline up to a total of 2 liters was infused over 30 to 60 minutes. A blood pressure cuff could be placed on the bag and inflated to 100 mm Hg to speed the flow. Patients were placed in a right side down, left side down, Trendelenburg and reverse Trendelenburg positions for 15 minutes (each position), in order to distribute the study medication through the peritoneal cavity.

If there was difficulty instilling the full 2 liters of fluid (due to patient discomfort or small size), the volume was decreased to 1.5 liters. If pain during instillation persisted despite a decrease in volume and adjustment of analgesic medications, the total volume was decreased to 1 liter. The peritoneal cavity was not drained after instillation. Only fluid for pharmacokinetic analysis was removed. At the end of the procedure, the peritoneal tubing/catheter was flushed with 5 to 10 mL of heparinized saline (100 U/mL). The patient's tolerance to the procedure and any concomitant medications administered (i.e. analgesics for pain) were noted. The appearance of the skin around the exit site, and any problems accessing the port site were noted. The actual start and completion times of the intraperitoneal study medication infusion were recorded. The actual volume of the IP infusion solution was recorded.

IP nanoparticle paclitaxel was administered in an open label dose escalation approach which utilized a modified accelerated titration method. The dose levels and cycle schedule are shown:

| Dose Level | Dose (mg/m2) | Day | Interval |
|---|---|---|---|
| 1 | 50 | 1 | q28 days |
| 2 | 82.5 | 1 | q28 days |
| 3 | 125 | 1 | q28 days |
| 4 | 175 | 1 | q28 days |
| 5 | 225 | 1 | q28 days |
| 6 | 275 | 1 | q28 days |

Two phases were utilized in the dose escalation procedure:

Acceleration Phase: an accelerated dose escalation scheme until one DLT occurred during cycle 1, followed by a standard dose escalation (3+3 design) based on CTCAE V3 toxicities was used. One subject per dose was treated until a subject had a dose limiting toxicity (DLT) or until a Grade 2 or higher non-hematological toxicity or Grade 3 or higher hematological toxicity occurred during the first cycle of treatment. At the time of this occurrence, the dosing was switched to the standard phase.

Standard Phase: At the time of the switch, two additional subjects were accrued at the level that triggered the switch. If only 1/3 of the subjects at this level experienced a DLT, then the next cohort of three subjects are treated at the next higher level. If 2/3 or 3/3 of the subjects experienced a DLT, then the next cohort of three subjects were enrolled at the next lower level.

At subsequent iterations, if;
0/3 of the subjects at a given level experienced a DLT, then the next cohort of three subjects was enrolled at the next higher dose level, if
1/3 of the subjects at a given level experience a DLT, then the next cohort of three subjects was enrolled at the same dose level, if
2/3 or 3/3 of the subjects at a given level experienced a DLT, then the next cohort of three subjects was enrolled at the next lower dose level,

If two of more of six subjects at a given level experienced a DLT, then the MTD had been exceeded, and another cohort of three subjects was treated at the next lower dose as explained above. The MTD was the highest dose for which the incidence of DLT was less than 33%. The recommended dose (RD) was defined as the highest dose level at which less than 1/3 evaluable subjects experienced a DLT.

Patient eligibility was determined as follows:

### Inclusion:

histologic or cytologic diagnosis of carcinoma predominantly confined to the peritoneal cavity
failure all potentially curative therapy and no other systemic treatment options available for
extra-peritoneal disease
at least 28 days elapsed since completion of previous chemotherapy treatment
a Zubrod Performance Status of 0-2
pretreatment granulocyte count >1,500/μl and platelet count >100,000/μl
adequate renal function documented by serum creatinine <1.5× the institutional upper limit of normal
adequate hepatic function documented by bilirubin of <2× the institutional upper limit of
normal and a SGOT <5× the institutional upper limit of normal
at least 18 years of age
may have received prior abdominal surgery >2 weeks prior to registration and have
recovered from all effects of the procedure

### Exclusion:

active inflammatory bowel disease or chronic diarrhea
known brain metastases
uncontrolled hypertension, unstable angina, symptomatic congestive heart failure, myocardial
infarction within previous 6 months or serious uncontrolled cardiac arrhythmia
concomitant radiation therapy, hormonal therapy, or other chemotherapy
active infection, requiring systemic therapy
concomitant medications demonstrated to inhibit or induce CYP3A4 or CYP2C8
Grade 2 or greater sensory neuropathy (by NCI Common Toxicity Criteria)
pregnant or nursing women
pre-existing conditions that prohibit the use of IV dexamethasone

Subjects participated in the study for a maximum of 27 months. Thirteen subjects (61.9%) had ovarian cancers, and one subject each (4.8%) had cancers of the bladder, brain, endometrium, gastroesophageal junction, pancreas, peritoneum, small bowel, or not specified.

### Pharmacokinetic Assessments:

Serial peritoneal and blood samples were collected for pharmacokinetic analysis of paclitaxel concentrations in systemic circulation and in the peritoneal fluid. Samples were collected prior to administration and at predetermined time points up to 14 days after administration. paclitaxel concentrations were determined by the method of Mortier et al. (Analytical Chemistry 2005; 77: 4677-4683) by LC-MS/MS.

Peritoneal fluid and blood samples for determination of paclitaxel concentrations were collected over a 336 hour period following the initiation of an intraperitoneal infusion of the study drug for the first treatment cycle (Day 1, Week 1) and a second treatment cycle (Day 1, Week 5), if appropriate.

Peritoneal fluid samples for determination of paclitaxel concentrations were collected over a 336-hour period following the initiation of the intraperitoneal infusion of study drug for the first and second treatment cycles, if appropriate. Peritoneal samples for pharmacokinetic analysis were attempted prior to study drug administration and at 2, 6, 8, 24, 48, 72, 168 (7 days), and 336 (14 days) hours. Plasma samples for pharmacokinetic analysis were collected prior to study drug administration and at 0.5, 1, 2, 4, 6, 8, 24, 48, 72, 168 (7 days), and 336 (14 days) hours.

### Pharmacodynamic Assessments:

Subjects' performance status was evaluated at the end of every treatment cycle and at follow up (i.e. every 4 weeks if treatment continued) using the Zubrod performance scale; and tumor response assessed at for treatment cycles 1, 2, 4, 5, 6, and follow up using evaluations consistent with RECIST guidelines.

Pharmacokinetic results are depicted in the figures.

### Example 8

A concentrated suspension containing paclitaxel particles can be prepared with the following ingredients in the amounts indicated.

| INGREDIENT | AMOUNT | |
|---|---|---|
| paclitaxel particles | 0.1%-10% | wt. |
| Water | Q.S. to 100% | wt. |

One or more excipients not listed above can be added to the powder of paclitaxel particles, to the liquid carrier or to the suspension. The solid ingredients are mixed and then placed in water to form a suspension. Other non-solid ingredients are added to the suspension as needed. The suspension is diluted as needed prior to administration to achieve a concentration of paclitaxel particles suitable for IP administration.

### Example 9

A suspension containing paclitaxel particles can be prepared with the following ingredients in the amounts indicated.

| INGREDIENT | AMOUNT | |
|---|---|---|
| paclitaxel particles | 0.05 to 10% | wt. |
| Buffer, to pH 6-7.5 | 0.01-0.5M | |
| Water | Q.S. to 100% | wt. |
| Tonicity adjusting agent | 0.1 to 1% | wt. |
| Demulcent | 0-1% | wt. |
| Viscosity modifier | 0-1% | wt. |
| Surfactant | 0-1% | wt. |

### Example 10

A suspension containing paclitaxel particles can be prepared with the following ingredients in the amounts indicated.

| INGREDIENT | AMOUNT (% WT.) |
|---|---|
| paclitaxel particles | 0.3-0.6% |
| Phosphate buffer, to pH 6.8-7.4 | 0.05-0.15M |
| NaCl | 0.5-1.0% |
| Water | Q.S. to 100% |
| Surfactant | 0-0.1% |
| Demulcent | 0-1% |
| Viscosity modifier | 0-1% |

### Example 11

A suspension containing paclitaxel particles can be prepared with the following ingredients in the amounts indicated.

| INGREDIENT | AMOUNT (% WT.) |
|---|---|
| paclitaxel particles | 0.4-0.6% |
| Phosphate buffer | 0.09-0.12M |
| NaCl | 0.9% |
| Polysorbate 80 | 0-0.1% |
| Water | Q.S. to 100% |
| Demulcent | 0-1% wt. |
| Osmotic agent | 0-1% wt. |

### Example 12

A suspension containing paclitaxel particles can be prepared with the following ingredients in the amounts indicated.

| INGREDIENT | AMOUNT (% WT.) | |
|---|---|---|
| Paclitaxel particles (average particle size in | 0.5-0.75% | wt |
| the range of 0.6-1.0 microns) | | |
| Sodium Phosphate (pH 6.8-7.4) | 0.01M | |
| NaCl | 0.75-0.9% | wt. |
| Polysorbate 80 | 0.1% | wt. |
| Water | Q.S. to 100% | wt. |

As used herein, the term “about” is taken to mean±10%, ±5%, ±2.5% or ±1% of a respective value.

As used herein, the terms “comprising”, “consisting essentially of” and “consisting of” can be used interchangeably unless otherwise indicated.

The above is a detailed description of particular embodiments of the invention. It will be appreciated that, although specific embodiments of the invention have been described herein for purposes of illustration, various modifications may be made without departing from the spirit and scope of the invention. Accordingly, the invention is not limited except as by the appended claims. All of the embodiments disclosed and claimed herein can be made and executed without undue experimentation in light of the present disclosure.

1-4.) (canceled)

5) A method of treating a tumor comprising:
administering intraperitoneally to a human subject with a tumor in the peritoneal cavity a suspension comprising paclitaxel particles and an aqueous carrier, wherein the paclitaxel particle comprises at least 95% paclitaxel, wherein the dose of paclitaxel is between 50 mg/m² and 275 mg/m², and wherein the ratio of peak paclitaxel concentration in intraperitoneal fluid to peak paclitaxel systemic concentration is between approximately 375 and approximately 2000.

6) The method of claim 5, wherein the paclitaxel particles are uncoated and exclude polymer, protein, polyethoxylated castor oil and polyethylene glycol glycerides composed of mono-, di- and triglycerides and mono- and diesters of polyethylene glycol.

7) The method of claim 5, wherein the tumor is selected from the group consisting of a breast tumor, an ovarian tumor, a lung tumor, a bladder tumor, and a prostate tumor.

8) The method of claim 7, wherein the suspension further comprises one or more components selected from the group consisting of polysorbate, methylcellulose, polyvinylpyrrolidone, mannitol, and hydroxypropyl methylcellulose.

9) The method of claim 5, wherein the paclitaxel particles have an average particle size in the range of 0.5-1.5 microns by number distribution.

10) The method of claim 9, wherein the paclitaxel particles have a size distribution as follows: D10 of 0.4 microns and a D90 of 1.5 microns by number distribution.

11) The method of claim 5, wherein the dose of paclitaxel is between 82.5 mg/m² and 275 mg/m².

12) The method of claim 5, wherein the dose of paclitaxel is between 175 mg/m² and 275 mg/m².

13) The method of claim 5, wherein the tumor is an ovarian tumor.

14-16.) (canceled)

17) The method of claim 5, wherein the suspension is administered by perfusion or as a bolus into the peritoneal cavity.

18-24.) (canceled)

25) The method of claim 5, wherein administration is initiated after removal of ascites fluid from the peritoneal cavity.

26-37.) (canceled)
